(19) United States
(12) Patent Application Publication
(10) Pub. No.: US 2006/0235364 A1
(43) Pub. Date: Oct. 19, 2006

(54) Needle-less fluid delivery assembly and error prevention system

(75) Inventors: Bridget M. O'Hare, Livonia, MI (US); Ali Y. Farhat, Dearborn Heights, MI (US)

Correspondence Address: CLARK HILL, P.C., 500 WOODWARD AVENUE, SUITE 3500, DETROIT, MI, 48226, US

(73) Assignee: Wizard MED, LLC, Canton, MI (US)

(21) Appl. No.: 11/104,912

(22) Filed: Apr. 13, 2005

(51) Int. Cl. A61M 5/32; A61B 19/00; A61M 5/00

(52) U.S. Cl. 604/411; 604/187

(57) ABSTRACT

A fluid delivery assembly transfers fluid from a first fluid-filled container filled to a second fluid container. The fluid delivery assembly includes a tube extending through a tube length between a base end which is fixedly secured to one of the first and second fluid containers and a distal end. The tube has an inner surface defining a tube contour. The fluid delivery assembly also includes a mating yoke secured to the other containers. The mating yoke defines a body periphery correlating to the tube contour such that the mating yoke in the one fluid containers is received within the tube and is capable of moving along the tube length until the first and second fluid containers are in fluid communication. Another aspect of this invention is it incorporates a container which eliminates the need for an exposed needle to draw the medicine out of the container.

### BACKGROUND ART

1. Field of the Invention

The invention relates to the delivery of fluids between storage containers. More particularly, the invention relates to a fluid delivery assembly for moving liquids between storage containers wherein the fluid delivery assembly is safe for the health care professional and the patient.

2. Description of the Related Art

Advancements in medicine are responsible for the treatment of many illnesses that were once thought untreatable. Some of the treatments require the parenteral administration of fluids using a syringe or an IV. As these opportunities to successfully treat patients grows, so do the number of medicines and protocols required for these treatments. More treatments expose healthcare professionals to accidental needle sticks which may result in the infection of the healthcare professionals. This problem is compounded by having increased pressures to maximize the productivity of the facilities and increasing acuity of patient illness, especially with the increases in nursing shortages. One result of this activity is more healthcare professionals acting in a hurried fashion which increases the risk for accidental needle sticks.

An additional problem to the increased medication protocols and the high frequency of patients entering a medical facility for treatment is that mistakes may be made in the administering of the medications to patients. More specifically, a patient may be properly diagnosed to receive a specific medicine and the patient may inadvertently receive a different medication because there was a simple mistake in the selection of the medicine when the healthcare professional was reaching for the medicine. Currently, all medicines come in the similarly shaped bottles, or bags in the case of an IV, and it is easy to accidentally select the wrong bottle, especially when an emergency procedure is being performed. Compounding this problem is the fact that all syringes are made of the same shape. This facilitates the ability for every syringe to be able to receive medication from any bottle, albeit different sizes. This creates a false sense of security that the contents therein are what is indicated on the label. More specifically, there is a high reliance on the belief that the syringe was filled correctly.

There have been several attempts to minimize each of the above-mentioned issues. In a U.S. Patent application having Ser. No. 10/392,519, a syringe protocol is disclosed wherein the syringe plunger is coded with a specific color, text, printed pattern or any combination of those codes. This aids a healthcare professional when administering medicines to a patient by having a readily visible code on the syringe to identify the contents of the syringe. This reference also discloses the use of bar code readers and RF tags to identify the type of syringe, which is based on the type of medicine it is storing therein. While this reference discloses a syringe that aids the healthcare professional in identifying what type of medicine is being injected into a patient, it provides little aid to the healthcare professional that is filling the syringe with the medicine. The person that fills the syringe will have the duty to make sure that the proper medicine is going into the properly coded syringe. Failure to do so will potentially cause even greater risks to the patient because healthcare professionals that receive the syringe will more readily make the assumption that the contents in the syringe appropriately match the coding on the plunger thereof. This may provide a false sense of security in the type of medicine being stored in the syringe by total reliance on human vigilance.

In U.S. Pat. No. 6,599,273, a fluid transfer device is shown wherein a needleless syringe is able to have medication transferred to it from a bottle. In FIGS. 6 and 7 of the patent, it is shown how a syringe is forced through a seal cap over an inverted needle that is fixedly secured to the bottle. Once the plunger is pulled to draw fluid into the barrel, the syringe may be removed therefrom. This design has a couple of flaws which renders it unsuitable for use in the medical field. First, the seal as designed may not return to a position covering the needle of the fluid transfer device. This will expose the needle to accidental pokes and while these accidental pokes may not result in the transfer of an infection, they will render the transfer device unsuitable for multiple use. Another problem associated with the transfer device is that it requires a needle to inject the bottle with which it is to be associated. As designed, the needle is too long and will prevent usage of some of the medicine because the needle extends too far down into the bottle rendering that portion of the medication irretrievable. Both will unnecessarily increase health care costs.

### SUMMARY OF THE INVENTION

A fluid delivery assembly transfers fluid from a first container filled with a fluid to a second fluid container. The fluid delivery assembly includes a tube extending through a tube length between a base end which is fixedly secured to one of the first and second fluid containers and a distal end. The tube has an inner surface defining a tube contour. The fluid delivery assembly also includes a mating yoke fixedly secured to the other of the first and second fluid containers. The mating yoke defines a body periphery correlating to the tube contour such that the mating yoke in the other of the first and second fluid containers is received within the tube and is capable of moving along the tube length until the first and second fluid containers are in fluid communication.

### BRIEF DESCRIPTION OF THE DRAWINGS

Advantages of the invention will be readily appreciated as the same becomes better understood by reference to the following detailed description when considered in connection with the accompanying drawings, wherein:

FIG. 1 is a side view of the invention incorporated into a bottle with a syringe about to enter the invention;

FIG. 1a is a cross-sectional side view of one embodiment of a modified syringe;

FIG. 2 is an exploded, perspective view of the invention and a syringe, partially cut away;

FIG. 3 is a side view of the bottle and one embodiment of the invention, with the invention shown in cross section;

FIG. 4 is a side view of a bottle, partially cutaway, and the invention, shown in cross section, with a syringe inserted therein;

FIG. 5 is an exploded, perspective view of the invention with a different shaped tube and mating yoke;

FIG. 6 is a top view of a bottle incorporating a second embodiment of the invention

FIG. 7 is a cross-sectional side view of a third embodiment of the invention with the syringe being engaged on top;

FIG. 8 is a cross-sectional side view of the third embodiment of the invention with a syringe being inserted therein;

FIG. 9 is a cross-sectional side view of the third embodiment of the invention with a modified syringe inserted therein;

FIGS. 10 through 13 are exploded, perspective views of several different shapes used in the embodiments of the invention; and

FIG. 14 is a syringe incorporating an alternative embodiment of the force function yoke.

### DETAILED DESCRIPTION OF THE PREFERRED EMBODIMENTS

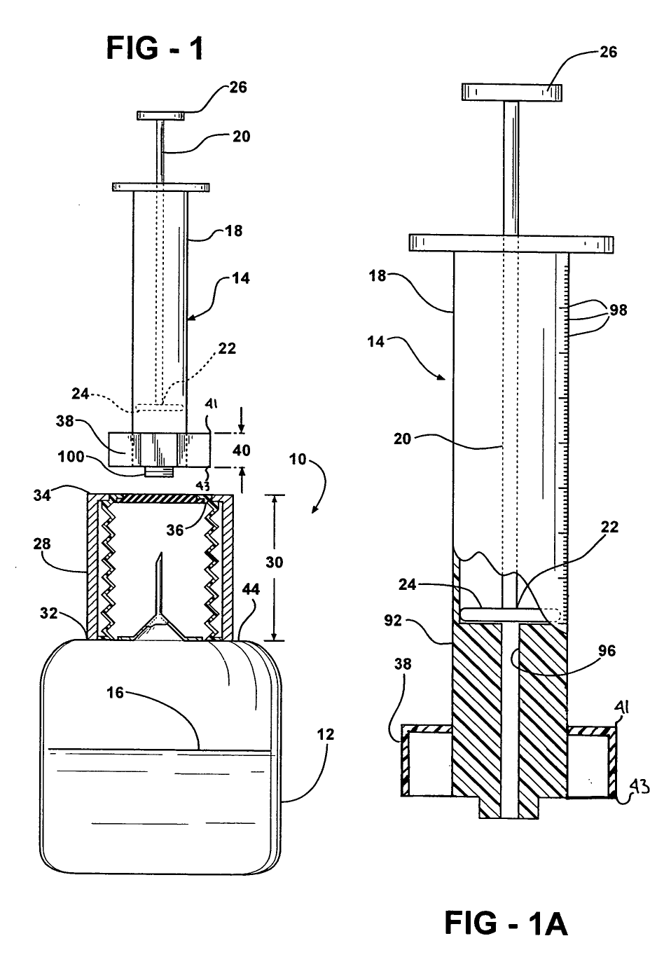

Referring to FIG. 1, a side view of a fluid delivery assembly is generally indicated at 10. The fluid delivery assembly is designed to transfer fluid from a first container 12 to a second container 14. In the embodiment shown in FIG. 1, the first container 12 is storing the fluid 16 that is to be transferred to the second container 14. For purposes of discussion with regard to FIG. 1, the first container is a medicine bottle containing liquid medicine and the second container 14 is a syringe. It should be appreciated by those skilled in the art that the liquid may be any type of liquid that a health care professional may need to use. A non-exhaustive list of liquids include medicines, blood and other bodily fluids, saline solutions, nutritional supplements and the like.

The syringe 14 includes a barrel 18 and a plunger 20. A first end 22 of the plunger 20 has a rubber stopper 24 secured thereto. A second end 26 of the plunger 20 is a thumbpress designed to receive a force in one of two directions to force the plunger 20 back out of the barrel 18 to draw medicine therein, and down through the barrel 18 when it is time to remove the fluid 16 from the syringe 14, into a redrawing container or patient.

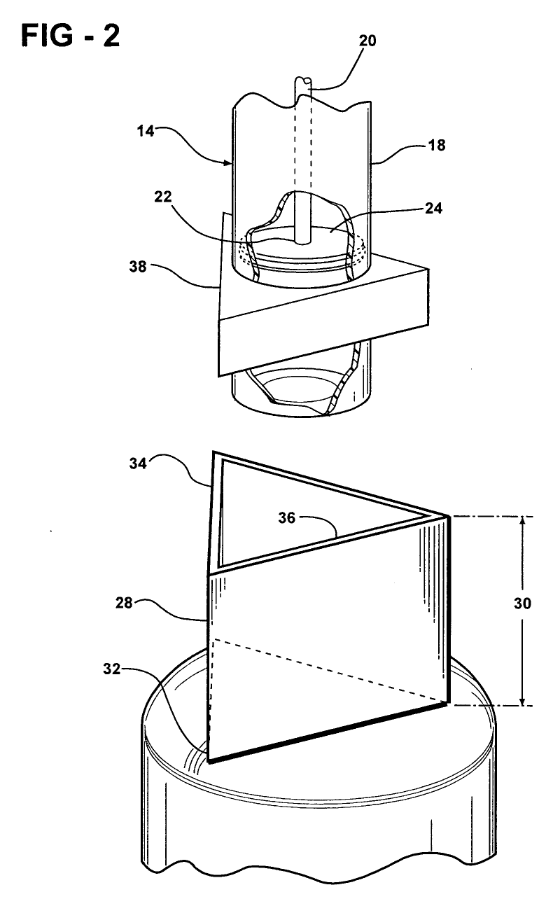

Referring to FIGS. 1 and 2, the fluid delivery assembly 10 includes a tube 28 extending through a tube length 30 between a base end 32 and a distal end 34. The base end 32 is fixedly secured to the first container 12 or an extension of the first container 12. The tube 28 defines an inner tube surface 36 that defines a tube contour. The fluid delivery assembly 10 also includes a mating yoke 38. The mating yoke 38 is fixedly secured to the syringe 14. The mating yoke defines a body periphery that is correlated to the tube contour such that the mating yoke 38 and the syringe 14 are received within the tube 28 a sufficient distance such that fluid 16 in the first container 12 may be transferred from the first container 12 to the syringe 14. This happens because the two containers 12, 14 are in fluid communication. The reason why the two containers 12, 14 are in fluid communication is because the body periphery of the mating yoke 38 is correlated to the inner surface 36 of the tube 28. If the two do not match or correlate, the mating yoke 38 will be prevented from entering the tube 28. This will ensure that the proper fluid 16 or medication is taken.

The mating yoke 38 and the tube 28 will be different for each category of medicine so that an incorrect medicine is not given. It is contemplated that a minimum of twelve different medicines or categories of medicines will be categorized such that each mating yoke 38 in tube 28 will have their own unique shape preventing a particular syringe 14 from being used improperly. Many shapes are shown in the Figures. It should be obvious to those skilled in the art that any number of shapes can be used with the invention and that those shown herein are exemplary of different possibilities. It is in no way an implication that these are the only shapes contemplated to be associated with the invention.

To further distinguish the syringes 14, color coding and pre-labeling allow the color coordination of the syringes with particular types of medicine that are to be used. The combination of the tube 28 and mating yoke 38 with the color coded syringe categories further ensures that the proper medication will be given and that a syringe is not pre-filled with a medicine that is not desired or improper to be given to a patient. It should be appreciated by those skilled in the art that RFID tagging and bar coding are two additional systems that may be used in augmenting the efficacy of the protocol associated with the invention. Many other systems or protocols may be incorporated without detracting from the invention as disclosed herein.

Because there are situations where there might be an urgency that fosters a desire of a healthcare professional to override the protocol, the mating yoke 38 defines a yoke length 40, extending from an upper edge 41 and a lower edge 43, which is less than one quarter of the tube length 30, which is less distance than is needed to reach a position to transfer the medicine. By having the yoke length 40 be substantially smaller than the tube length 30, the healthcare professional would be required to seek the proper syringe 14 for the proper first container 12. More specifically, the lengths 30, 40 are chosen such that it would be nearly impossible for a mating yoke 38 to be forced through the whole length of the tube 28 when the body periphery does not match the tube contour. To do so would most likely force the syringe off axis preventing the syringe from receiving the medicine 16.

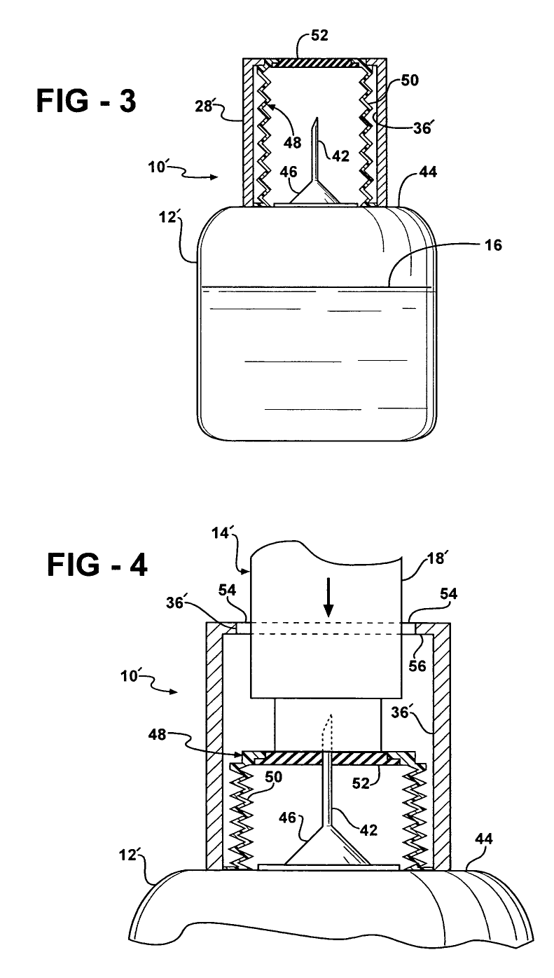

Referring to FIGS. 3 and 4, an alternative embodiment of the fluid delivery assembly is generally indicated at 10′, wherein all like primed numerals represent similar elements of the first embodiment, discussed above. In this second embodiment, a hollow needle 42 is fixedly secured to a top surface 44 of the first container 12′, which is a bottle. A funnel 46 extends between the needle 42 and the top surface 44 of the bottle 12′. The funnel 46 allows all of the fluid 16 in the bottle 12′ to be able to be removed through the needle 42 into a syringe 14′ once it is forced down over the needle 42 through the tube 28′.

Additionally in this second embodiment, there is a seal, generally indicated at 48. The seal 48 includes an accordion sheath 50 and a rubber cap 52. Cilia 54 extend between an opening 56 and the barrel 18′ of the syringe 14′. The cilia 54 helps maintain the inner surface 36′ of the tube 28′ clear of any debris that might be associated with the entrance of the barrel 18′ into the tube 28′. The rubber cap 52 is pierceable by the needle 42 when the syringe 14′ is inserted into the tube 28′ and forced down over the needle 42. Once the syringe 14′ is inserted sufficiently within the tube 28′, the syringe 14′ and the bottle 12′ are inverted and the plunger 20 is retracted from the barrel 18 to pull the fluid 16 out of the bottle 12′ into the barrel 18′ of the syringe 14′, as is done with standard medication transfer from a bottle into a syringe.

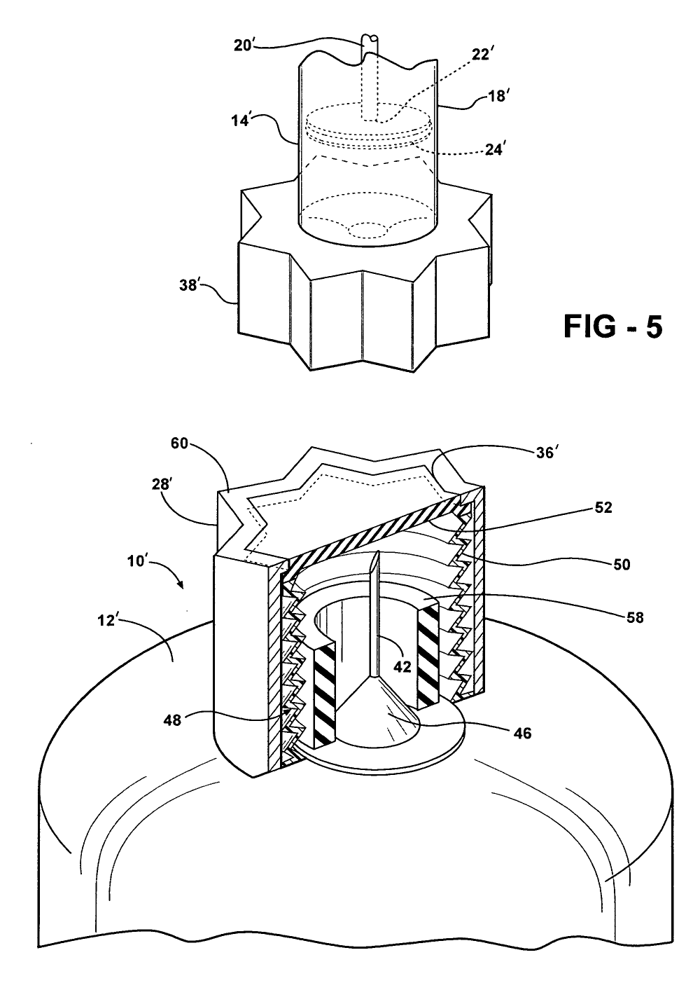

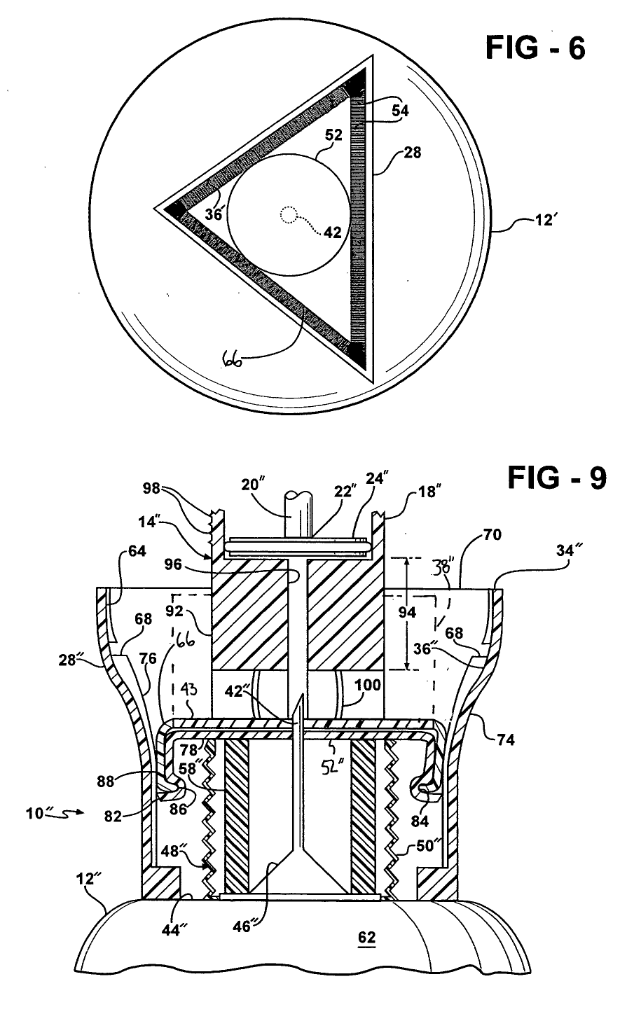

Referring to FIG. 5, a perspective view of the second embodiment is shown. In this figure, a stop 58 extends around the needle 42 to prevent the syringe 14′ from moving further into the tube 28′ than is necessary. In this embodiment, it is shown that the tube 28′ has an outer contour 60 that matches the inner surface 36′ of the tube 28′. The outer contour 60 matches in shape to facilitate the tactile features of the invention. Namely, a healthcare professional will be able to better choose the proper bottle 12′ if the healthcare professional can feel the outer contour 60 as well as see the outer contour 60.

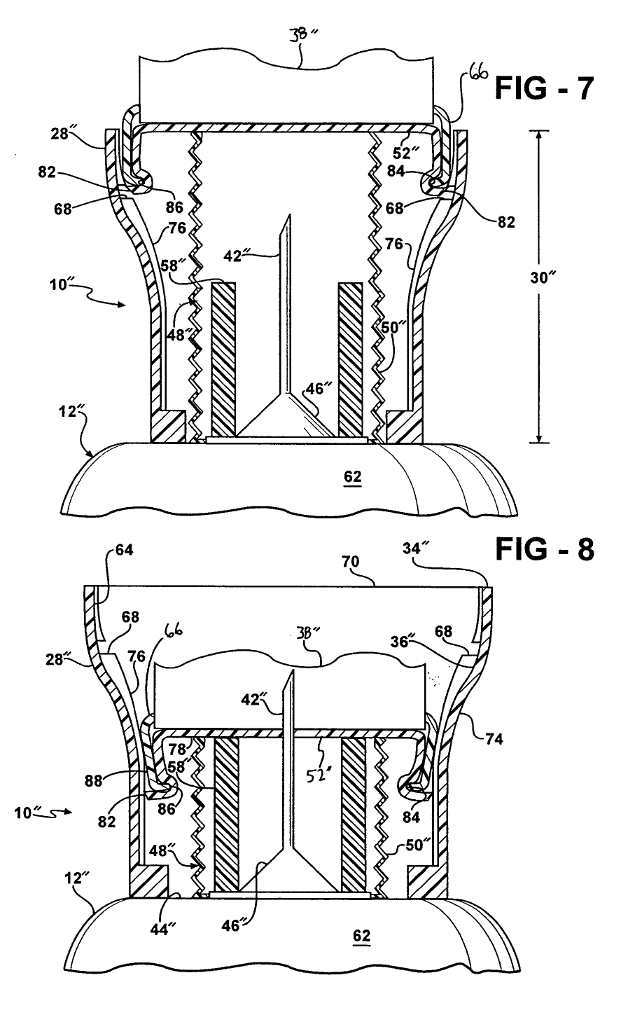

Referring to FIGS. 7 through 9, a third embodiment of the invention is generally shown at 10″, wherein double primed reference characters refer to similar elements as those in the first two embodiments. The fluid delivery assembly 10″ transfers fluid to a fluid receiving container, namely the syringe 14″. It should be appreciated that a device other than a syringe 14″ may be used with the fluid delivery assembly 10″. The fluid delivery assembly 10″ is fixedly secured to the fluid holding container, namely the first container 12″ or an extension of the first container 12″. The fluid holding container 12″ defines a reservoir 62 and a top surface 44″. The top surface 44″ covers the reservoir 62.

The tube 28″ extends out from the top surface to a neck opening 64. The tube 28″ defines an inner tube surface 36″ and defines the tube length 30″. The fluid delivery assembly 10″ includes a shape margin, generally shown at 66, which extends out of the lower edge 43″ of the mating yoke 38″. The shape margin 66 will be discussed in greater detail subsequently.

The fluid delivery assembly 10″ also includes a locking surface 68 that extends out from the inner tube surface 36″. The locking surface 68 prevents the rubber cap 52″ from sliding down the tube 28″.

The tube 28″ defines a distal perimeter 70 at the distal end 34″ thereof. The tube 28″ also defines a base perimeter 72 at the base end 32″. The distal perimeter 70 is similar in shape to the base perimeter 72. More specifically, if the distal perimeter 70 is a square, so too is the base perimeter 72. The distal perimeter 70 is, however, larger than the base perimeter 72. The tube 28″ includes a transition section 74 wherein the perimeter of the tube 28″ decreases between the distal perimeter 70 and the base perimeter 72 where the transition section 74 begins just above the tip of the needle 42. The transition section 74 has a gradual slope so as to not create unnecessarily sharp edges.

Returning attention to the locking surface 68, it is disposed within the tube 28″ and extends out from the inner tube surface 36″ substantially perpendicular thereto. The locking surface 68 is disposed along the tube 28″ generally at the beginning of the transition section 74 of the tube 28″. The transition section 74 is a taper between the distal end 34″ and the base end 32″. The locking surface 68 extends around the entire inner tube surface 36″. An extension 76 is disposed adjacent the locking surface 68 and extends down along a portion of the tube length 30″.

A locking cover 78 is omega-shaped in cross section and defines a locking flange 82 that extends out from the perimeter thereof. The locking cover 78 includes or extends out from the rubber cap 52″. When positioned in the lock position (FIG. 7), the locking flange 82 prevents the shape margin 66 from moving down into the tube 28″ because the locking flange 82 abuts against the locking surface 68. The locking cover 78 also includes a channel 84 that extends around the locking cover 78. The channel 84 is disposed adjacent the locking flange 82. The taper section 74 of the tube 28″ is designed to force the mating shape margin 66 to move as one piece (when the shape margin 66 is moved up and down the tube 28″). When the locking cover 78 and rubber cap 52″ are above the needle 42″, and the locking flange 82 is above the locking surface, the locking cover 78 and the shape margin 66 will separate. This will ensure that the needle 42″ will remain sterile for future multi-use purposes.

The extension 76 extends along the inner tube surface 36″ and is used to prevent the locking flange 82 from relocking prior to its movement above the locking surface 68, which is defined as having the rubber cap 52″ in a position such that the needle 42″ is covered thereby in its locked position due to its engagement of the locking flange 82 with the locking surface 68. This will ensure the rubber cap 52″ will be able to move to a position covering the needle 42″ after fluid 16 is transferred.

The shape margin 66 is generally C-shaped in cross section. Its dimensions conform substantially similar to the locking cover 78 in that the shape margin 66 and the locking cover 78 engage each other through much of their respectively adjoining surfaces. The shape margin 66 includes a lip 86 that is turned inwardly and nested within the channel 84. Therefore, the lip 86 and the channel 84 are matingly engaged with each other. The shape margin 66 defines an outer periphery 88 that extends along a tube portion thereof. The outer periphery 88 abuts the inner tube surface 36″ as well as the extensions 76. By having the outer periphery 88 abut the inner tube surface 36″, movement of the shape margin 66 downwardly forces the lip 86 into the channel 84. This moves the locking flange 82 inwardly and away from the locking surface 68 allowing the shape margin 66 to move downwardly within the tube 28″. It should be appreciated that the tube 28″ and the shape margin 66 may be fabricated from plastic impregnated with lubricating oils to facilitate in the reduction of force required to move the syringe 14″ into a position where it can receive fluid 16 therefrom. Should it be determined necessary, the tube 28″ can include bearings set therewithin to further reduce the force required to move the shape margin 66 with respect to the tube 28″.

Turning attention to the syringe assembly 14″ in FIG. 9 (with the mating yoke 38″ shown in phantom), it is shown to have a spacer 92 having a spacer length 94. The spacer 92 is at the first end 22″ of the barrel 18″. The spacer 92 includes a center hole 96 that allows fluid to move from the needle 42″ up into the barrel 18″. The spacer 92 prevents fluid 16 from being stored inside the barrel 18″ immediately adjacent the dispensing end, i.e., the first end 22″. The spacer 92 is put in place to allow a healthcare professional to draw fluid 16 from the bottle 12″ while the syringe 14″ is partially within the tube 28″. The spacer 92 does so by preventing fluid from being drawn into that space of the syringe 14″ allowing the healthcare professional to see graduations 98 which are spaced along the barrel 18″ starting at a distance away from the dispensing end 22″ a distance equal to the spacer length 94′ to reflect the volume inside the barrel 18″ consumed by the spacer 94″, less the stop 58″ length. The syringe assembly 14″ also includes an attachment portion 100 that is typically threaded to threadedly engage a needle or to subsequently dispense the fluid 16 from the syringe 14″.

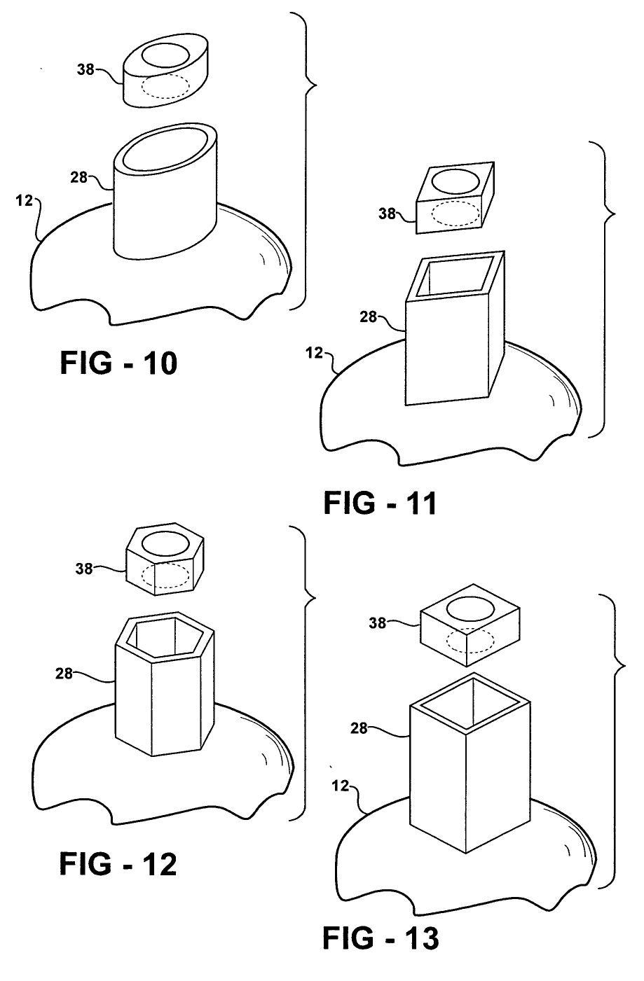

Referring to FIGS. 10 through 13, alternative embodiments of the force fit features of the invention are generally indicated wherein the mating yoke 38 and the tube 28 are shown in different physical configurations. It should be appreciated that there are several different shapes which may be included to distinguish the numerous medication or categories of medication types. The figures shown herein are but a few examples of the many shapes that may be utilized to perform the force fit function.

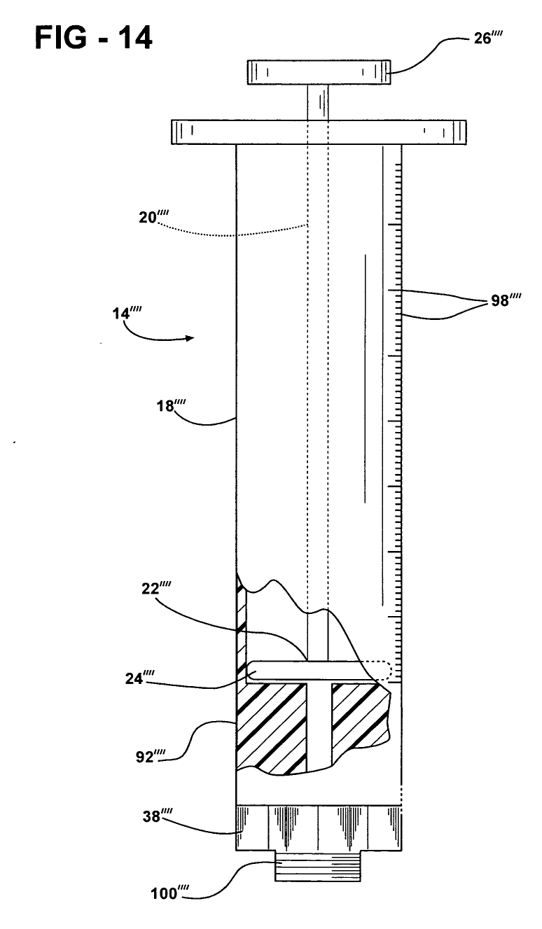

Referring to FIG. 14, an alternative embodiment of a syringe assembly 14″″ is shown wherein quadruple primed numerals represent like elements. The spacer 92″″ is disposed at the first end 22″″. The yoke 38″″ extends along the barrel 18″″ at the end of the spacer 92″″. The yoke 38″″ extends through a width that is equal to a diameter of the barrel 18″″. This design is contemplated for smaller syringe assemblies 14″″ to help ensure the syringe assembly 14″″ does not become to lopsided with much of the weight of the syringe assembly 14″″ being at one end thereof.

The invention has been described in an illustrative manner. It is to be understood that the terminology, which has been used, is intended to be in the nature of words of description rather than of limitation.

Many modifications and variations of the invention are possible in light of the above teachings. Therefore, within the scope of the appended claims, the invention may be practiced other than as specifically described.

We claim:

1. A fluid delivery assembly for delivering fluid from a first container filled with a fluid to a second fluid container, said fluid delivery assembly comprising:
a tube extending through a tube length between a base end, fixedly secured to one of the first and second fluid containers, and a distal end, said tube having an inner surface defining a tube contour; and
a mating yoke fixedly secured to the other of the first and second fluid containers, said mating yoke defining a body periphery correlating to said tube contour such that said mating yoke and the other of the first and second fluid containers is received within said tube and is capable of moving along said tube length until the first and second fluid containers are in fluid communication.

2. A fluid delivery assembly as set forth in claim 1 wherein said mating yoke defines a yoke length less than one quarter said tube length.

3. A fluid delivery assembly for delivering fluid to a fluid receiving container, said fluid delivery assembly comprising:
a fluid holding container defining a reservoir and a top surface covering said reservoir;
a tube extending out from said top surface to a tube opening, said tube defining an inner tube surface and a tube length;
a tube cover movably secured to said tube to cover said tube opening, said tube cover slidable along said tube length; and
a locking surface extending out from said inner tube surface preventing said tube cover from sliding down said tube.

4. A fluid delivery assembly as set forth in claim 3 including an unlock extension disposed adjacent said locking surface and extending along a portion of said tube length.

5. A fluid delivery assembly as set forth in claim 4 wherein said tube cover includes a locking cover and an unlocking cover matingly engaging said locking cover.

6. A fluid delivery assembly as set forth in claim 5 wherein said locking cover includes a locking flange engagable with said locking surface to prevent movement of said neck cover along said neck.

7. A fluid delivery assembly as set forth in claim 6 wherein said locking cover includes a channel extending around said locking cover and disposed adjacent said locking flange.

8. A fluid delivery assembly as set forth in claim 7 wherein said unlocking cover includes a lip extending inwardly to matingly engage said channel of said locking cover.

9. A fluid delivery assembly as set forth in claim 8 wherein said unlocking cover defines an outer periphery abutting said inner tube surface such that movement of said tube cover downwardly forces said lip into said channel and moves said locking flange inwardly and away from said locking surface allowing said tube cover to move downwardly with said tube.

10. A fluid delivery assembly as set forth in claim 9 wherein said outer periphery includes engagement protrusions increasing dimensions of said outer periphery.

11. A fluid delivery assembly as set forth in claim 10 including a needle fixedly secured to said top surface of said fluid holding container.

12. A fluid delivery assembly as set forth in claim 11 wherein said needle extending upwardly through a portion of said neck to a pointed end.

13. A fluid delivery assembly as set forth in claim 12 wherein said neck length is longer than said needle.

14. A fluid delivery assembly as set forth in claim 13 including a seal covering said needle.

15. A fluid delivery assembly as set forth in claim 14 wherein said needle cover defines a thickness such that said needle cover is pierceable by said pointed end of said needle as said needle cover slides down said neck.

16. A fluid delivery assembly as set forth in claim 15 including a funnel extending between said top surface of said fluid holding container and said needle to facilitate the flow of the fluid inside said fluid holding container to said needle.

17. A fluid delivery assembly as set forth in claim 3 wherein said tube includes a transition section along said tube length wherein said inner tube surface is smaller therebelow.

18. A syringe assembly for delivering fluid between containers, said syringe assembly comprising:
a barrel for receiving and retaining fluids therein; said barrel having a handle end and a dispensing end;
a plunger slidably engaging said barrel to draw fluids into said barrel and to force the fluids out therefrom; and
a spacer defining a spacer length disposed with said barrel at said dispensing end to prevent the fluid from being stored inside said barrel immediately adjacent said dispensing end.

19. A syringe assembly as set forth in claim 18 including graduations along said barrel starting a distance away from said dispensing end equal to said spacer length to reflect volume inside said barrel consumed by said spacer.

20. A syringe assembly as set forth in claim 19 including a mating yoke disposed around said barrel covering said spacer, wherein said mating yoke defines a shape specific to a medicine category or medicine.
